(19) United States
(12) Patent Application Publication
(10) Pub. No.: US 2024/0207206 A1
(43) Pub. Date: Jun. 27, 2024

(54) Improved Topical Composition of Colchicine

(71) Applicant: Zenvision Pharma LLP, Mumbai (IN)

(72) Inventors: Sivakumar Venkata Bobba, Navi Mumbai (IN); Bhimrao Jadhav, Navi Mumbai (IN); Dhananjay Shinde, Navi Mumbai (IN); Sunil Pophale, Navi Mumbai (IN); Shankar Pol, Navi Mumbai (IN)

(21) Appl. No.: 17/768,113

(22) PCT Filed: Oct. 7, 2020
(86) PCT No.: PCT/IB2020/059395

§ 371 Date: Apr. 11, 2022

(30) Foreign Application Priority Data
Oct. 11, 2019 (IN) 201921041165

(51) Int. Cl. A61K 31/165; A61K 9/00; A61K 9/06

(52) U.S. Cl. CPC A61K 31/165; A61K 9/0014; A61K 9/06

(57) ABSTRACT

The present invention relates to improved topical composition of Colchicine or a pharmaceutically acceptable salt thereof as an active agent and, process of preparing such improved topical composition. The invention also relates to the use of said improved topical composition for prophylaxis and the treatment of acute gout flares.

### FIELD OF THE INVENTION

The present invention relates to a pharmaceutical composition comprising Colchicine or a pharmaceutically acceptable salt thereof as an active agent, process of preparation thereof and method of using the same.

The present invention relates to an improved topical composition of Colchicine or a pharmaceutically acceptable salt thereof, process of preparing such improved topical composition and the use of said improved topical composition for prophylaxis and the treatment of acute gout flares.

### BACKGROUND OF THE INVENTION

A gout flare is an intensely painful and disabling inflammatory arthritis, usually involving a single joint but occasionally involving two or more joints. Gout attacks, or flares, are due to persistently elevated levels of uric acid in the blood. The elevated levels cause needle-like crystals to form in joint and the surrounding tissue, causing pain, swelling, and redness. Gout affects about 1 to 2% of the Western population at some point in their lives. This is believed to be due to increasing risk factors in the population, such as metabolic syndrome, longer life expectancy, and changes in diet. Older males are most commonly affected. Although flares can be quite painful, medication can help control gout and limit flares.

The majority of gout cases are treated with medication. Medication can be used to treat the symptoms of gout attacks, prevent future flares, and reduce the risk of gout complications such as kidney stones and the development of tophi. Commonly used medications include nonsteroidal anti-inflammatory drugs (NSAIDs), colchicine, or corticosteroids. These reduce inflammation and pains in the areas affected by gout and are usually taken orally. Medications can also be used to either reduce the production of uric acid (xanthine oxidase inhibitors such as Allopurinol) or improve the kidney's ability to remove uric acid from the body (Probenecid).

Colchicine is an alkaloid chemically described as (S)N-(5,6,7,9-tetrahydro-1,2,3, 10-tetramethoxy-9-oxobenzo[alpha] heptalen-7-yl) acetamide with a molecular weight of 399.4, a molecular formula of C₂₂H₂₅NO₆ and Colchicine is represented by compound of structural formula I.

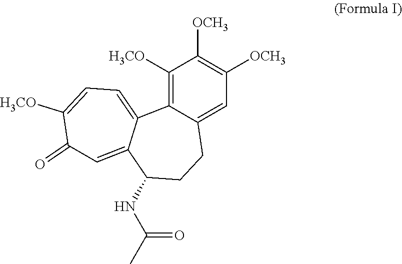 [SMILES: COc1cc2c(c(OC)c1OC)-c1ccc(OC)c(=O)cc1[C@@H](NC(C)=O)CC2]

Colchicine occurs as a pale yellow powder that is soluble in water.

U.S. Patent Publication number 20170119707 discloses homeopathic topical composition (gel) of Colchicine for prophylaxis and the treatment of acute gout flares. The said patent publication does not disclose stable nanoemulsion based topical composition of Colchicine.

U.S. Patent Publication number 20170348211 discloses topical Colchicine alone or in combination with in combination with pentoxifylline and tocopherol (Vitamin E) composition in the form of suspension, emulsion, solution, gel, paste, ointment, cream, lotion, spray or aerosol. The said patent publication does not disclose stable, nanoemulsion based topical composition of Colchicine.

Chinese Publication number CN102366403 discloses topical microemulsion composition of Colchicine. The said patent publication does not disclose nanoemulsion stable based topical composition of Colchicine.

CN103251550 discloses topical cream and ointment composition of Colchicine. The said patent does not disclose stable nanoemulsion based topical composition of Colchicine.

The PCT Publication number WO2007129162 (A2) discloses topical emulgel composition of a derivative of colchicine and a substantially lipophilic compound having pharmacological activity. The said published PCT does not disclose nanoemulsion based topical composition of Colchicine.

There are different topical pharmaceutical compositions of Colchicine are available in the prior art but said compositions have drawbacks such as low penetration, larger particle size of drugs that are difficult to penetrate, poor bioavailability, greasiness of composition, difficulty of application, storage and stability issues, possibility of allergenic reactions and the like. These drawbacks result in low efficacy and subsequently poor patient compliance for prophylaxis and the treatment of acute gout flares.

Hence, there is still need to design an improved topical composition of Colchicine that has low particle size of drug, enhanced penetration, increased bioavailability, prolonged stability, efficacy and reduced side effects.

The inventors of the present invention surprisingly found that a topical pharmaceutical composition of Colchicine or a pharmaceutically acceptable salt thereof with penetration enhancer Poly ethylene glycol (PEG) ester surfactant, Diethylene glycol monoethyl ether and other pharmaceutically acceptable excipients provides an improved topical composition of Colchicine, wherein said composition possess properties such as improved penetration, high therapeutic efficiency, storage stability and better patient compliance for prophylaxis and the treatment of acute gout flares.

### OBJECTS OF THE INVENTION

It is an object of the present invention to provide a pharmaceutical composition comprising Colchicine or a pharmaceutically acceptable salt thereof and at least one pharmaceutically acceptable excipient.

It is another object of the present invention to provide an improved topical composition comprising Colchicine or a pharmaceutically acceptable salt thereof and at least one pharmaceutically acceptable excipient including penetration enhancer or surfactants, preservative, antioxidant, vehicle, gelling agent, thickening agent, cosurfactant, humectant, acidifying or alkalizing or buffering agent, absorbent, chelating agent, opacifying agent in a sufficient concentration to provide a stable composition.

It is another object of the present invention provides an improved topical composition comprising:
a) Colchicine or a pharmaceutically acceptable salt thereof;
b) Penetration enhancer; and
c) Optionally other suitable pharmaceutically acceptable excipients.

Wherein penetration enhancer are selected from the group consisting of Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02, Gelucire® 54/02, Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 and the like.

It is another object of the present invention to provide an improved topical composition comprising:
a) 0.001% to 10% w/w of Colchicine or a pharmaceutically acceptable salt thereof;
b) 0.5 to 30% w/w of penetration enhancer or surfactant;
c) Optionally other suitable pharmaceutically acceptable excipients.

It is another object of the present invention to provide an improved topical composition comprising:
a) 0.001% to 10% w/w of Colchicine or a pharmaceutically acceptable salt thereof;
b) 0.5 to 30% w/w of penetration enhancer or surfactant;
c) Optionally other suitable pharmaceutically acceptable excipients;
Wherein said improved topical composition provides better penetration and storage stability; and
Wherein said improved topical composition is in the form of cream or gel.

It is another object of an invention to provide improved stable topical composition of Colchicine or a pharmaceutically acceptable salt thereof; wherein the composition is in the form of nanoemulsion based cream or nanoemulsion based gel.

It is another object of an invention to provide a process for the preparation of an improved topical composition, wherein process comprises steps of:
a) Preparing aqueous phase consisting of Colchicine or a pharmaceutically acceptable salt thereof;
b) Preparing lipid phase consisting of other suitable pharmaceutically acceptable excipients;
c) Emulsifying lipid phase of step (b) to aqueous phase of step (a) to form an improved topical composition

It is another object, there is provided a method of using an improved topical composition of Colchicine or a pharmaceutically acceptable salt thereof for prophylaxis and the treatment of acute gout flares.

### SUMMARY OF THE INVENTION

In one aspect, an invention provides a pharmaceutical composition comprising Colchicine or a pharmaceutically acceptable salt thereof and at least one pharmaceutically acceptable excipient.

In another aspect, the present invention provides an improved topical composition comprising Colchicine or a pharmaceutically acceptable salt thereof and at least one pharmaceutically acceptable excipient including penetration enhancer or surfactants, preservative, antioxidant, vehicle, gelling agent, thickening agent, cosurfactant, humectant, acidifying or alkalizing or buffering agent, absorbent, chelating agent, opacifying agent in a sufficient concentration to provide a stable composition.

In another aspect the present invention provides an improved topical composition comprising:
a) Colchicine or a pharmaceutically acceptable salt thereof;
b) Penetration enhancer; and
c) Optionally other suitable pharmaceutically acceptable excipients.

Wherein penetration enhancer are selected from the group consisting of Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02, Gelucire® 54/02, Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 and the like.

In another aspect, an invention provides an improved topical composition comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients.

In another aspect, the present invention provides an improved topical composition comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition provides better penetration and storage stability.

In another aspect, the present invention provides an improved topical composition in the form of cream comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition in the form of cream provides better penetration and storage stability.

In another aspect, the present invention provides an improved topical composition in the form of gel comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition in the form of gel provides better penetration and storage stability.

In another aspect, the present invention provides improved stable topical composition of Colchicine or a pharmaceutically acceptable salt thereof; wherein the composition is in the form of nanoemulsion based cream.

In another aspect, the present invention provides improved stable topical composition of Colchicine or a pharmaceutically acceptable salt thereof; wherein the composition is in the form of nanoemulsion based gel.

In another aspect, the present invention provides an improved topical composition comprising:
a) 0.001% to 10% w/w of Colchicine or a pharmaceutically acceptable salt thereof;
b) 0.5 to 30% w/w of at least one penetration enhancer;
c) Optionally other suitable pharmaceutically acceptable excipients;
Wherein said improved topical composition provides better penetration and storage stability; and Wherein said at least one penetration enhancer is selected from the group consisting of Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02, Gelucire® 54/02, Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 and the like.

Another aspect of an invention provides process for the preparation of an improved topical composition, wherein process comprises:
a) Preparing aqueous phase consisting of Colchicine or a pharmaceutically acceptable salt thereof;
b) Preparing lipid phase consisting of other suitable pharmaceutically acceptable excipients; and
c) Emulsifying lipid phase of step (b) to aqueous phase of step (a) to form an improved topical composition.

In preferred aspect, there is provided a method of using an improved topical composition of Colchicine or pharmaceutically acceptable salt thereof for prophylaxis and the treatment of acute gout flares.

### DETAIL DESCRIPTION OF THE INVENTION

The term “composition”, as in topical pharmaceutical composition, is intended to encompass a drug product comprising Colchicine or its pharmaceutically acceptable salts thereof, and other inert ingredient(s) (pharmaceutically acceptable excipients).

The terms like “topical pharmaceutical composition” or “improved topical composition” or “topical composition” or “composition” or “dosage form” are synonymous and have same meaning in context of present invention.

The term “topical pharmaceutical composition” is used in a broad sense; it includes application of medication to a particular place on or in the body. Most often topical administration means application to body surfaces such as the skin or mucous membranes to treat ailments via a large range of classes including creams, foams, gels, lotions, and ointments.

Preferably, the topical pharmaceutical composition refers to cream or gel. More preferably, the topical pharmaceutical composition refers to an improved topical composition comprising nanoemulsion based cream or gel which provides better penetration and storage stability

The term “Colchicine” is used in broad sense to include not only “Colchicine” per se but also its pharmaceutically acceptable salts, solvates, hydrates, enantiomers, derivatives, isomers, polymorphs, prodrugs thereof. The amount of Colchicine according to the invention may be present at 0.001% to 10%, by weight based on total weight of the composition, preferably, 0.01% to 5% w/w. The amount of Colchicine ranges from 0.01 mg to 100 mg; preferably 0.1 mg to 20 mg; more preferably 0.1 mg to 10 mg.

The term “pharmaceutically acceptable salt” refers to salts derived from a variety of organic and inorganic counter ions including fumarate, maleate, phosphate, L-tartrate, citrate, acetate, oxalate, and sulfate or the like.

The term “excipient”, means a pharmacologically inactive component such as penetration enhancer or surfactants, preservative, antioxidant, vehicle, gelling agent, thickening agent, cosurfactant, humectant, acidifying or alkalizing or buffering agent, absorbent, chelating agent, opacifying agent or the like. The excipients that are useful in preparing a pharmaceutical composition are generally safe, non-toxic and are acceptable for veterinary as well as human pharmaceutical use. Reference to an excipient includes both one and more than one such excipient

The term, “Nanoemulsion”, is a colloidal particulate system in the submicron size range acting as carriers of drug molecules which are thermodynamically stable, composed of two immiscible liquids mixed along with emulsifying agents (surfactants and co-surfactants) to form a single phase. Their size varies from 10 to 1,000 nm.

The term “Nanoemulsion based gel” or “Nanoemulsion based cream” according to present invention means cream or gel formulation which comprises Nanoemulsion.

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

The term “stable” as used herein refers to formulations that substantially retain the label amount of the therapeutically active ingredient during storage for commercially relevant times, and the drug-related impurity contents in the formulations remain within the acceptable limit.

The terms like “stable” or “storage stability” are synonymous and have same meaning in context of present invention.

Throughout this specification and the appended claims, it is to be understood that the words “comprise”, “have”, “contain” and “include” and variations such as “comprises”, “comprising”, “having”, “containing” “includes”, “including” are to be interpreted inclusively, unless the context requires otherwise. That is, the use of these words may imply the inclusion of an element or elements not specifically recited.

The example of bases or emulsifying agent includes but not limited to Cetostearyl alcohol, Stearyl alcohol, Carnauba wax, Cetyl alcohol, Cetyl ester wax, Emulsifying wax, Hydrous lanolin, Lanolin, Lanolin alcohols, Vegetable oils and Animal fat; Coconut oil, Bees wax, Olive oil, Spermaceti wax, Sesame oil, Almond oil, Alcohols, Acids and Esters; oleic acid, Oleyl alcohol, Palmitic acid, Lauryl alcohol, Lauric acid, Myristyl alcohol, Ethyl oleate, Isopropyl myristate, Ethylene glycol, Hydrogenated and Sulphated oils; castor oil, corn oil, evening primrose oil, linseed oil, mineral oil, peanut oil, clove oil, propylene dicaprylate/dicaprate glycol, glyceryl tricaprylate, and triglycerides (LCT, MCT, SCT), Hydrogenated castor, Cotton seed, Hydrogenated sulphated castor oils, Microcrystalline wax, Liquid Paraffin, Petrolatum, Propylene glycol, Polyethylene glycol, Stearic acid, Natural gums like acacia and tragacanth, hydrophilic colloids such as acacia and finely divided solids, e.g., bentonite and veegum, monovalent and bivalent soaps, cholesterol or cholesterol esters, triethanolamine and its salts, dodecyl benzene sulfonate, diethylene glycol monoethyl ether and docusate sodium or combinations thereof. The amount of base or Emulsifying agent present in the composition ranges from about 0.02-40%; preferably about 0.1-30% by weight of composition.

The example of Penetration enhancer includes but not limited to Poly ethylene glycol (PEG) ester surfactant, Diethylene glycol monoethyl ether, isopropyl myristate, polysorbate 80, sorbitan monooleate, propylene glycol, ethanol, isopropyl alcohol, oleic acid, polyethylene glycol, phospholipids, Cyclodextrins, black pepper (Piper nigrum), Pyrrolidones, dimethyl sulphoxide (DMSO), Decylmethyl sulphoxide (DCMS), terpenes (cineole, eugenol, d-limonene, menthol, menthone), urea, polyethylene glycol monolaurate, and butanediol; ethers, including diethylene glycol mono ethyl ether and diethylene glycol monomethyl ether; fatty acids, including lauric acid, oleic acid, and valeric acid; fatty acid esters, isopropyl palmitate, methyl propionate, and ethyl oleate; dimethyl acetamide, dimethylformamide 2-pyrrolidone, ethanolamine, methyl-2-pyrrolidone, diethanolamine, and triethanolamine; alkanones; organic acids, including salicylic acid, citric acid, and succinic acid; or any mixtures thereof. surfactants like spans and tweens, labrasol, labrafil, cremophor, poloxamer, sorbitan monostearate, sodium lauryl sulfate, propylene glycol monostearate, gelucire, natural gums like acacia and tragacanth, hydrophilic colloids such as acacia and finely divided solids, e.g., bentonite and veegum, monovalent and bivalent soaps, cholesterol or cholesterol esters, triethanolamine and its salts, dodecyl benzene sulfonate, docusate sodium or combinations thereof. The penetration enhancer is present in the composition in an amount of about 0.05% to 50% based on the total weight of the composition; preferably in an amount of about 0.5% to 30%, more preferably 5 to 25% based on the total weight of the composition.

Preferably, the examples of penetration enhancers according to present invention include but not limited to Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02. Gelucire® 54/02. Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 or mixture thereof. Preferably, the concentration of Poly ethylene glycol (PEG) ester surfactant ranges from 0.5 to 20% based on the total weight of the composition and concentration of Diethylene glycol monoethyl ether ranges from 5 to 25% based on the total weight of the composition.

The examples of Preservatives includes but not limited to methyl paraben, chorocresol, benzoic acid, phenyl mercuric nitrate, benzyl alcohol, benzoic acid and its salts, boric acid, methyl paraben, propyl paraben, trihydrate and anhydrous sodium acetate, chlorhexidine, formaldehyde, glutaraldehyde, imidazolidinyl urea, trichlosan, benzalkonium chloride and chloroxylenol or combination thereof. The preservative is present in the composition in an amount of about 0.001% to 5% based on the total weight of the composition; preferably in an amount of about 0.05% to 2% based on the total weight of the composition.

The examples of Antioxidant includes but not limited to butylated hydroxyl toluene, butylated hydroxyl anisole, propyl gallate, polyols like sorbitol, xylitol and maltitol, natural extracts like quillaia, or lactic acid or urea, lithium chloride, ascorbic acid, sodium metabisulfite, carotinoids, carotenes such as α-carotone, β-carotene and lycopene, chlorogenic acid, tocopherols, uric acid, ZnO and ZnSO4 or combination thereof. The Antioxidant is present in the composition in an amount of about 0.001% to 5% based on the total weight of the composition; preferably about 0.005-1.0% based on the total weight of the composition

The examples of Vehicle includes but not limited to purified water, medium chain triglycerides, propylene glycol, hexylene glycol, oleyl alcohol, propylene carbonate, mineral oil, almond oil, cottonseed oil, ethyl oleate, isopropyl myristate, isopropyl palmitate, myristyl alcohol, octyldodecanol, olive oil, peanut oil, safflower oil, sesame oil, soybean oil and squalene or combination thereof. The Vehicle is present in the composition in an amount of about 5% to 70% based on the total weight of the composition; preferably in an amount of about 10% to 50% based on the total weight of the composition.

The examples of Gelling agent includes but not limited to Carbomer/Carbopols, Pemulen®, cellulose derivatives such as Methyl Cellulose, Hydroxy Ethylcellulose, Hydroxy Propylmethylcellulose, Carboxy methyl cellulose, Hydroxy propyl cellulose, etc., Glycerol, or Propylene glycol gelled with suitable agents such as natural gums such as Xanthan gum and Tragacanth, Fenugreek Mucilage, Pectin, Poloxamers (Pluronics), Alginate, Gelatin, Starch, Polyvinyl alcohol, Povidone or combination thereof.

The examples of Thickening agent include but not limited to carbomer, hydrogenated castor oil, methyl cellulose, sodium carboxyl methyl cellulose, carrageenan, colloidal silicon dioxide, natural gum such as gelatin, tragacanth gum and guar gum, hydroxypropyl cellulose, hydroxypropyl methyl cellulose, polyethylene oxide, alginic acid, paraffin, cetostearyl alcohol, PEG 200, PEG 300, PEG 400, PEG 600, monoethanolamine, triethanolamine, glycerol, propylene glycol, polyoxyethylene sorbiton monoleate, and poloxamers, polyvinyl pyrrolidone, various alcohols such as polyvinyl alcohol, ethanol or isopropyl alcohol, fumed silica or combination thereof.

The examples of Cosurfactant according to present invention include but not limited to transcutol, ethylene glycol, propylene glycol, ethanol, isopropanol, propylene glycol, glycerin, and PEG 400, dimethyl sulphoxide (DMSO) and dimethyl acetamide (DMA) or combinations thereof.

The examples of Humectant includes but not limited to glycerin, glyceryl triacetate, propylene glycol, polyethylene glycol, sorbitol solution, 1,2,6 hexanetriol, isopropyl myristate, petrolatum, isopropyl palmitate, hydrogenated castor oil, mineral oil, polyoxymethylene urea and potassium sorbate or combination thereof.

The examples of Acidifying or Alkalizing or buffering agent includes but not limited to anhydrous and monohydrate citric acid, phosphoric acid, sodium hydroxide, potassium hydroxide, sodium bicarbonate, potassium sorbate, monobasic and dibasic sodium phosphate and trolamine or combination thereof.

The examples of Absorbent include but not limited to magnesium carbonate, calcium carbonate, starch, cellulose and its derivatives or combination thereof.

The examples of Chelating agent includes but not limited to ethylene diamine tetraacetate, dimercaprol, dimercaptosuccinic acid, penicillamine, deferoxamine, deferasirox, citric acid, maleic acid, phosphoric acid and like or combination thereof.

The examples of Opacifying agent include but not limited to higher fatty alcohols such as cetyl, stearyl, cetostearyl alcohol, arachidyl and behenyl alcohols, solid esters such as cetyl palmitate, glyceryl laurate, various fatty acid derivatives such as propylene glycol and polyethylene glycol esters, inorganic materials such as, magnesium aluminum silicate, zinc oxide, titanium dioxide or other sunblocking agents.

In one embodiment, an invention relates to a pharmaceutical composition comprising Colchicine or pharmaceutically acceptable salt thereof and at least one pharmaceutically acceptable excipient.

In another embodiment, the invention relates to an improved topical composition comprising Colchicine or pharmaceutically acceptable salt thereof and at least one pharmaceutically acceptable excipient including penetration enhancer, preservative, antioxidant, vehicle, gelling agent, thickening agent, cosurfactant, humectant, acidifying or alkalizing or buffering agent, absorbent, chelating agent, opacifying agent in a sufficient concentration to provide a stable composition.

In another embodiment, an invention provides an improved topical composition comprising:
a) Colchicine or a pharmaceutically acceptable salt thereof;
b) Penetration enhancer; and
c) Optionally other suitable pharmaceutically acceptable excipients.

Wherein penetration enhancer is selected from the group consisting of Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02, Gelucire® 54/02, Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 and the like.

In another embodiment, an invention provides an improved topical composition comprising:
a) Colchicine or a pharmaceutically acceptable salt thereof;
b) Penetration enhancer;
c) antioxidant;
d) vehicle; and
Wherein said improved topical composition provides better penetration and storage stability.

In another embodiment, an invention provides an improved topical composition comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition provides better penetration and storage stability.

In another embodiment, an invention provides an improved topical composition in the form of cream comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition in the form of cream provides better penetration and storage stability.

In another embodiment, an invention provides an improved topical composition in the form of gel comprising:
a) 0.001% to 10% w/w of Colchicine or a pharmaceutically acceptable salt thereof;
b) 0.5 to 30% w/w of penetration enhancer;
c) Optionally other suitable pharmaceutically acceptable excipients; and
Wherein said improved topical composition in the form of gel provides better penetration and storage stability.

In another embodiment, an invention provides improved stable topical composition of Colchicine or a pharmaceutically acceptable salt thereof; wherein the composition is in the form of nanoemulsion based cream.

In another embodiment, an invention provides improved stable topical composition of Colchicine or a pharmaceutically acceptable salt thereof; wherein the composition is in the form of nanoemulsion based gel.

In another embodiment, an invention provides an improved topical composition comprising:
a) 0.001% to 10% w/w of Colchicine or a pharmaceutically acceptable salt thereof;
b) 0.5 to 30% w/w of at least one penetration enhancer;
c) optionally other suitable pharmaceutically acceptable excipients;
Wherein said improved topical composition provides better penetration and storage stability; and
Wherein said at least one penetration enhancer is selected from the group consisting of Poly ethylene glycol (PEG) ester surfactant (Gelucire® 48/16, Gelucire® 50/13, Gelucire® 44/14, Gelucire® 48/16, Gelucire® 55/18, Gelucire® 35/10, Gelucire® 48/09, Gelucire® 43/01, Gelucire® 39/01, Gelucire® 33/01, Gelucire® 50/02, Gelucire® 54/02, Gelucire® 64/02) or other brands of Poly ethylene glycol (PEG) ester surfactant, Diethylene glycol monoethyl ether (Transcutol®), Isopropyl myristate, Sorbitan monooleate, Polysorbate 80 and the like.

In another embodiment, Colchicine according to present invention treats gout flares by blocking neutrophil-mediated inflammatory responses induced by monosodium urate crystals in synovial fluid. Colchicine disrupts the polymerization of ß-tubulin into microtubules, thereby preventing the activation, degranulation and migration of neutrophils to sites of inflammation. Colchicine also interferes with the inflammasome complex found in neutrophils and monocytes that mediates interleukin-1ß (IL-1B) activation.

In another embodiment, the improved stable topical composition of Colchicine cream or gel comprises one or more pharmaceutically acceptable excipients. The excipients according to present inventions are compatible and acceptable to the topical application.

The one or more pharmaceutically acceptable excipients may be selected from the group consisting of bases or emulsifying agent, penetration enhancer or surfactants, preservative, antioxidant, vehicle, gelling agent, thickening agent, cosurfactant, humectant, acidifying or alkalizing or buffering agent, absorbent, chelating agent, and opacifying agent etc.

In another embodiment, an invention provides the process for the preparation of an improved topical composition, wherein process comprises steps of:
a) Preparing aqueous phase consisting of Colchicine or a pharmaceutically acceptable salt thereof;
b) Preparing lipid phase consisting of other suitable pharmaceutically acceptable excipients; and
c) Emulsifying lipid phase of step (b) to aqueous phase of step (a) to form an improved topical composition.

In another embodiment, an invention provides the process for the preparation of an improved topical cream composition, wherein process comprises steps of:
a) Preparing aqueous phase consisting of Colchicine or a pharmaceutically acceptable salt thereof;
b) Preparing lipid phase consisting of other suitable pharmaceutically acceptable excipients; and
c) Emulsifying lipid phase of step (b) to aqueous phase of step (a) to form an improved topical composition.

In another embodiment, an invention provides the process for the preparation of an improved topical gel composition, wherein process comprises steps of:
a) Preparing aqueous phase consisting of Colchicine or a pharmaceutically acceptable salt thereof;
b) Preparing lipid phase consisting of other suitable pharmaceutically acceptable excipients; and
c) Emulsifying lipid phase of step (b) to aqueous phase of step (a) to form an improved topical composition.

In another embodiment, the improved stable topical composition of Colchicine or a pharmaceutically acceptable salt thereof of the present invention can be manufactured by any suitable method known in the art such as High energy emulsification method: high pressure homogenizer, ultrasonication and microfluidization; Low energy emulsification method: solvent evaporation technique, hydrogel method, Phase inversion emulsification method, spontaneous emulsification; Brute force method, trituration method, levigation method, fusion method, chemical reaction method, emulsification method, thermal change method, flocculation or by simply incorporation of an emulsion into the gel base.

In another embodiment, the concentration or amount of Colchicine or a pharmaceutically acceptable salt thereof and one or more pharmaceutically acceptable excipients, also the manufacturing process has been optimized in such way that topical Colchicine composition provides better penetration, no creaming, caking and sedimentation, ease of application and spreadability, prevention of greasiness, increased bioavailability, more efficacy and better patient compliance for prophylaxis and the treatment of acute gout flares.

In another embodiment, the improved stable topical composition of Colchicine or a pharmaceutically acceptable salt thereof according to present invention were evaluated for their organoleptic properties like color, odour and appearance; drug content uniformity, homogeneity, consistency, spreadability, phase behavior study, flocculation and creaming, cracking, particle size analysis/droplet size analysis, pH, assay, related substance and found to be in the compliance.

In another embodiment, the improved topical composition of Colchicine or a pharmaceutically acceptable salt thereof according to present invention were loaded for stability condition at 40° C.±2° C./75%±5% RH, 25° C.±2° C./60%±5% RH and 30° C.±2° C./65%±5% RH for six months and found to be stable. The assay and related substances data of the product were found to be in the compliance.

In another embodiment, the improved topical composition of Colchicine or a pharmaceutically acceptable salt thereof according to present invention is stable; provide better penetration, increased bioavailability, more efficacy and better patient compliance for prophylaxis and the treatment of acute gout flares.

In another embodiment, the improved stable topical composition of Colchicine or a pharmaceutically acceptable salt thereof according to present invention can be packaged into the suitable container like collapsible tubes, jars, pot, bottle or single dose packets or any suitable packaging material. The container material or packaging material of the present invention does not affect the quality of the preparation or does not allow diffusion of any kind into or across the material of the container into the preparation.

### EXAMPLES

The following Examples are provided solely for illustrative purposes and are not meant to limit the scope of the invention in any way.

### Example 1

### Compositions of Colchicine 0.01% Cream

| Sr. | | Quantity |
|---|---|---|
| No. | Ingredients | (g/100 g) |
| 1. | Colchicine | 0.01 |
| 2. | Medium chain triglycerides | 10.00 |
| 3. | Isopropyl myristate | 5.00 |
| 4. | Propylene glycol | 25.00 |
| 5. | Cetostearyl alcohol | 6.00 |
| 6. | Stearyl alcohol | 6.00 |
| 7. | Polysorbate 80 | 2.5 |
| 8. | Sorbitan monooleate (Span 80) | 2.5 |
| 9. | Methylparaben | 0.15 |
| 10. | Gelucire 48/16 | 5.00 |
| 11. | Butylated hydroxyl toluene | 0.01 |
| 12. | Purified water | q.s. to 100 g |

### Manufacturing Process:

1. Aqueous phase: purified water was warm heated at 42º−45° C., Polysorbate 80 was dissolved completely to it. Further water was heated at 65°−70° C., Colchicine was dissolved completely;
2. Lipid phase: Medium chain triglycerides, Isopropyl myristate, Propylene glycol, Cetostearyl alcohol, Stearyl alcohol, Sorbitan monooleate (Span 80), Methylparaben, Butylated hydroxyl toluene, Gelucire 48/16, heated at temperature 65°−70° C.;
3. Emulsification: lipid phase of step (2) to aqueous phase step (1) at 65°-70° C., further homogenized using IKA ultra turrex at 3000 rpm for 10 minutes;
4. Manufactured cream was further cooled down at room temperature using overhead stirrer for uniform cooling and mixing;
5. Weight make up was done by purified water.

### Physical Parameters (Example 1):

| Sr. | | |
|---|---|---|
| No. | Parameters | Observations |
| 1. | Appearance | Smooth white cream, slightly stiff |
| | | textured. |
| 2. | pH | 7.08 |
| 3. | Viscosity | 1,53,200 cps |
| | (SC4-34 spindle, 0.3 rpm) | |

### Stability Study Data Results (Example 1):

| | | | | Un- | |
|---|---|---|---|---|---|
| Sr. | | Time | % | known | Total |
| no. | Condition | point | Assay | Max | Impurities |
| 1 | Initial | — | 100.2 | ND | ND |
| 2 | 40° C. ± 2° C./75% ± 5% RH | 1 M | 102.0 | ND | ND |
| 3 | 40° C. ± 2° C./75% ± 5% RH | 2 M | 103.8 | ND | ND |
| 4 | 40° C. ± 2° C./75% ± 5% RH | 3 M | 102.9 | ND | ND |
| 5 | 40° C. ± 2° C./75% ± 5% RH | 6 M | 103.2 | ND | ND |
| 6 | 25° C. ± 2° C./60% ± 5% RH | 3 M | 103.5 | ND | ND |
| 7 | 25° C. ± 2° C./60% ± 5% RH | 6 M | 101.2 | ND | ND |
| 8 | 30° C. ± 2° C./65% ± 5% RH | 3 M | 102.0 | ND | ND |
| 9 | 30° C. ± 2° C./65% ± 5% RH | 6 M | 102.5 | ND | ND |

*ND = Not detected

### Example 2

### Compositions of Colchicine 0.01% Cream

| Sr. | | Quantity |
|---|---|---|
| No. | Ingredients | (g/100 g) |
| 1. | Colchicine | 0.01 |
| 2. | Medium chain triglycerides | 10.00 |
| 3. | Isopropyl myristate | 5.00 |
| 4. | Propylene glycol | 25.00 |
| 5. | Cetostearyl alcohol | 6.00 |
| 6. | Stearyl alcohol | 6.00 |
| 7. | Polysorbate 80 | 2.5 |
| 8. | Sorbitan monooleate (Span 80) | 2.5 |
| 9. | Methylparaben | 0.15 |
| 10. | Diethylene glycol monoethyl ether | 20.00 |
| | (Transcutol P) | |
| 11. | Butylated hydroxyl toluene | 0.01 |
| 12. | Purified water | q.s. to 100 g |

### Manufacturing Process:

1. Aqueous phase: purified water was warm heated at 42°−45° C. Polysorbate 80 was dissolved completely in it. Further water was heated at 65°−70° C., Colchicine was dissolved completely;
2. Lipid phase: Medium chain triglycerides, Isopropyl myristate, Propylene glycol, Cetostearyl alcohol, Stearyl alcohol, Sorbitan monooleate (Span 80), Methylparaben, Butylated hydroxyl toluene, Diethylene glycol monoethyl ether (Transcutol P), mixed and heated at temperature 65°−70° C.
3. Emulsification: lipid phase of step (2) mixed to aqueous phase step (1) at 65°-70° C., further homogenized using IKA ultra turrex at 3000 rpm for 10 minutes;
4. Manufactured cream was further cooled down at room temperature using overhead stirrer for uniform cooling and mixing;
5. Weight make up was done by purified water.

### Physical Parameters: (Example 2):

| Sr. | | |
|---|---|---|
| No. | Parameters | Observations |
| 1. | Appearance | Glossy white cream with smooth |
| | | texture |
| 2. | pH | 6.94 |
| 3. | Viscosity | 1,47,300 cps |
| | (SC4-34 spindle, 0.3 rpm) | |

### Stability Study Data Results: (Example 2)

| | | | | Un- | |
|---|---|---|---|---|---|
| Sr. | | Time | % | known | Total |
| no. | Condition | point | Assay | Max | Impurities |
| 1 | Initial | — | 100.1 | ND | ND |
| 2 | 40° C. ± 2° C./75% ± 5% RH | 1 M | 98.2 | ND | ND |
| 3 | 40° C. ± 2° C./75% ± 5% RH | 2 M | 98.0 | ND | ND |
| 4 | 40° C. ± 2° C./75% ± 5% RH | 3 M | 98.9 | ND | ND |
| 5 | 40° C. ± 2° C./75% ± 5% RH | 6 M | 100.1 | ND | ND |
| 6 | 25° C. ± 2° C./60% ± 5% RH | 3 M | 99.5 | ND | ND |
| 7 | 25° C. ± 2° C./60% ± 5% RH | 6 M | 97.5 | ND | ND |
| 8 | 30° C. ± 2° C./65% ± 5% RH | 3 M | 97.0 | ND | ND |
| 9 | 30° C. ± 2° C./65% ± 5% RH | 6 M | 98.4 | ND | ND |

*ND = Not detected

We claim:

1. A topical pharmaceutical composition comprising:
a) Colchicine or a pharmaceutically acceptable salt thereof;
b) a penetration enhancer;
c) optionally other suitable pharmaceutically acceptable excipients; and
wherein, penetration enhancer is selected from the group consisting of Poly ethylene glycol (PEG) ester surfactant, Diethylene glycol monoethyl ether or mixture thereof.

2. The topical pharmaceutical composition according to claim 1 is storage stable.

3. The topical pharmaceutical composition according to claim 1 is in the form of creams, gels, foams, lotions or ointments.

4. The topical pharmaceutical composition according to claim 1, further comprising penetration enhancer selected from the Isopropyl myristate, Sorbitan monooleate, Polysorbate 80 or mixture thereof.

5. The topical pharmaceutical composition according to claim 1, wherein concentration of Colchicine or a pharmaceutically acceptable salt thereof ranges from 0.001% to 10% by weight of the composition.

6. The topical pharmaceutical composition according to claim 1, wherein concentration of penetration enhancer ranges from 0.5 to 30% based on the total weight of the composition.

7. The topical pharmaceutical composition according to claim 1, wherein concentration of Poly ethylene glycol (PEG) ester surfactant ranges from 0.5 to 20% based on the total weight of the composition and concentration of Diethylene glycol monoethyl ether ranges from 5 to 25% based on the total weight of the composition.

8. The topical pharmaceutical composition according to claim 1, further comprises one or more pharmaceutically acceptable excipient selected from the group consisting of emulsifying agent, preservative, antioxidant, vehicle, gelling agent, thickening agent, cosurfactant, humectant, acidifying or alkalizing or buffering agent, absorbent, chelating agent, and opacifying agent or mixture thereof.

9. The topical pharmaceutical composition according to claim 8, wherein one or more pharmaceutically acceptable excipient are Cetostearyl alcohol, Stearyl alcohol, Methylparaben, Butylated hydroxyl toluene, Medium chain triglycerides, Propylene glycol and water or mixture thereof.

10. A process for the preparation of a topical composition, wherein process comprises steps of:
a) Preparing aqueous phase consisting of Colchicine or a pharmaceutically acceptable salt thereof;
b) Preparing lipid phase consisting of other suitable pharmaceutically acceptable excipients; and
c) Emulsifying lipid phase of step (b) to aqueous phase of step (a) to form an improved topical composition.
